(19) United States
(12) Patent Application Publication
(10) Pub. No.: US 2018/0232500 A1
(43) Pub. Date: Aug. 16, 2018

(54) TIERED CLASSIFICATION AND QUANTITATION SCHEME FOR MULTIVARIATE ANALYTICAL DATA

(71) Applicant: Savannah River Nuclear Solutions, LLC, Aiken, SC (US)

(72) Inventors: Robert J. Lascola, North Augusta, SC (US); Patrick E. O'Rourke, Martinez, GA (US); David Immel, Augusta, GA (US); Jean R. Plummer, Aiken, SC (US); Edward A. Kyser, III, Aiken, SC (US)

(73) Assignee: Savannah River Nuclear Solutions, LLC

(21) Appl. No.: 15/429,372

(22) Filed: Feb. 10, 2017

(51) Int. Cl. G06F 19/00; G01N 33/20; G01N 21/31

(52) U.S. Cl. CPC G06F 19/707; G01N 2201/12; G01N 21/31; G01N 33/20

(57) ABSTRACT

Analysis techniques by generation and interpretation of multivariate data that can provide for highly accurate analyte detection are described. Protocols can include a tiered principal component analysis (PCA) utilizing a partial least squares (PLS) approach for classification of a sample. Methods include selection of a particular local model for each classification category. The classification categories are determined based on assessment of sample characteristics such as solution absorbance, acidity, analyte oxidation state distribution, temperature, presence of one or more interferents, etc.

### STATEMENT REGARDING FEDERALLY SPONSORED RESEARCH OR DEVELOPMENT

This invention was made with government support under Contract No. DE-AC09-08SR22470 awarded by the U.S. Department of Energy. The government has certain rights in the invention.

### BACKGROUND

Industrial processing is often enhanced by in situ analysis. Benefits include increased processing speed and efficiency, enhanced worker safety, reduced waste, and real-time tracking of material inventory. Instruments used for the analysis may be of various types, but share the common property that they measure a signal that changes proportionally to the quantity of analyzed material that is in the sample.

It is often the case that the measurement of a sample is complicated by factors that can generate a change in the analytical signal but are otherwise uncorrelated to the quantity being measured. In these situations, it is necessary to distinguish the specific contributions of the analyte from the other factors for the measurement to be accurate. This is possible if multivariate data can be generated in which the contributions of the analyte and the extraneous factors are not correlated. Such data may be obtained by use of multiple instruments that are sensitive to distinct physical properties, by use of a single instrument that exhibits a distinct response at different values of the same property, or by some combination thereof. Types and properties of multivariate data are known in the art, for instance as described by Booksh, K. S. and Kowalski, B. R. “Theory of Analytical Chemistry”, Anal. Chem. (1994) 66(15), 782A-791A.

Examples of a single instrument that can exhibit a distinct response at different values of the same property include any one of several forms of optical spectroscopy, in which the response of the sample to different wavelengths of light can be measured. One form of optical spectroscopy that has wide utility in process analysis is absorbance spectroscopy. In this technique, the degree to which a sample will absorb or transmit light can be measured continuously and simultaneously at multiple wavelengths. This can allow for the application of sophisticated mathematical models to establish accurate determination of analyte presence or concentrations over a wide range of process conditions. For instance, absorbance spectroscopy is a useful tool for monitoring the chemical separation and purification of individual or mixed actinide sources as the actinides have distinctive spectra that are sensitive to oxidation state, complexation (e.g. with NO₃⁻ in nitric acid solutions), temperature, and ionic strength.

Unfortunately, there are difficulties in obtaining accurate analyte detection via absorbance spectroscopy, particularly when considering in situ applications. While the extra data obtainable by this technique can allow for statistical models that can compensate to a degree for the spectral variations described above, often the variations are highly correlated and the number of spectral factors required for these models can introduce extra uncertainty into a detection regime. This uncertainty can greatly reduce process throughput, for instance by requiring lower actinide concentrations to maintain conservatism with respect to criticality safety.

Due to such difficulties, absorbance-based detection strategies have been confined to either limited accuracy at a potentially broad range of conditions (e.g., in situ processing) or higher accuracy at highly defined conditions (e.g., off-line processing). The necessity of incorporating multivariate analyses into spectral analysis techniques has limited the application of in situ absorbance spectroscopy in many industrial processes including actinide processing due to this trade-off between detection accuracy and sample variability.

What are needed in the art are methods for analyzing multivariate data in general, and absorbance spectral data in particular, that can increase detection accuracy across a wide range of variability in sample conditions such as may be expected during in situ analysis.

### SUMMARY

Disclosed is a method for analyzing a sample through interpretation of multivariate data generated for the sample. More specifically disclosed is a tiered classification system that can provide high detection accuracy over a wide range of sample conditions such as total analyte concentration, the presence of analytes in different states (e.g., oxidation states), variable sample states (gas, liquid, solid, etc.), the presence of spectral interferents, sample temperature, sample acidity, etc.

A method can include assigning a sample into one of multiple classifications based upon a value of a first characteristic of the sample. In general, the value of the first characteristic can be determined through the interpretation of multivariate data, and values of the multivariate data can be sensitive to a concentration of an analyte contained in the sample and/or can be sensitive to a concentration of one or more additional components of the sample and/or can be sensitive to a property of the sample. For example, in one embodiment, the sample can be in the form of a solution and can be assigned into a classification based upon the absorbance of the solution at one or more particular wavelength ranges.

A method can also include further assigning the sample into one of multiple primary sub-classifications based upon a value of a second characteristic of the sample, the value of the second characteristic being determined through further interpretation of the multivariate data. Optionally, a method can include further categorizing the sample into one of multiple secondary sub-classifications based upon a value of a third characteristic of the sample. Following all desired categorizing of the sample, a method can include predicting the concentration of the analyte in the sample through application of one or more measurement models specific to the categorizations of classification, primary sub-classification and optional secondary sub-classification of the sample.

Significantly, the particular model used for each different classification and sub-classification need not be the same, and a sample can be categorized into each class and sub-class by use of a model particularly suited for that classification, e.g., a first model of acidity for a highly absorbent sample, a second, different model of acidity for a low absorbent sample, etc.

Beneficially, the multivariate data used in the classification and measurement models can be obtained by any suitable methodology and system. For example, the data procurement can be completely automated in one embodiment and the classifications and analyte determination can all be based upon spectral characteristics of the sample as determined by use of a spectrophotometer. According to another embodiment, the multivariate data can be generated through utilization of a plurality of instruments.

As each measurement model can be optimized to account for the reduced range of sample conditions associated with that classification and sub-classification, each model can be more accurate and less uncertain than a prediction model that attempts to address the entire range of solution conditions.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1A illustrates the variation in absorbance spectra for plutonium solutions with variation in the acidity of the solutions.

FIG. 1B illustrates the variation in absorbance spectra for plutonium solutions with variation in the relative amount of the plutonium at different oxidation states.

FIG. 1C illustrates the variation in absorbance spectra for plutonium solutions with variation in temperature of the solutions.

FIG. 2 illustrates a flowsheet as may be utilized in one embodiment of a method.

FIG. 3 presents a comparison of results obtained with global and local total Pu concentration models showing the effect of spectral noise.

FIG. 4 presents a comparison of results obtained with global and local total Pu concentration models showing the effect of interferents in the sample solution,

FIG. 5 presents PCs of Pu⁴⁺ (circles) and mixed Pu³⁺/Pu⁶⁺ (dotted line) obtained by rotation of PCs obtained by self-consistent PCA. The known spectrum for pure Pu⁴⁺ (solid line) is shown for comparison.

FIG. 6 presents an estimate of molar absorptivity for Pu⁶⁺ from analysis of disproportionated Pu standards.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to analysis techniques that can provide for highly accurate analyte detection and quantitation in a protocol that generates a multivariate data set. In one embodiment, the multivariate data set can include or consist entirely of an absorbance spectrum. However, application of the disclosed method to other types of multivariate data sets, whether generated by a single instrument or the combined analyses of multiple instruments, will be evident to those versed in the field.

In one embodiment, the analysis can encompass a tiered principal component analysis (PCA) type method that can utilize a partial least squares (PLS) approach for total analyte measurements in a processing stream. Rather than using a single global principal component (PC) model that covers all expected sample conditions as has been known in the past, the method selects a particular local model for each classification and/or sub-classification. The classifications and sub-classifications can be designated and differentiated from one another based on assessment of sample characteristics such as, and without limitation to, UV/Vis/IR characteristics (e.g., absorbance, reflectance, emission), acidity, analyte oxidation state distribution, temperature, presence of one or more interferents, etc. Specific characteristics will be apparent based upon the analytical method(s) used to generate the multivariate data set and the chemical and physical nature of the process and sample being monitored.

Beneficially, use of a plurality of individualized local classification models can provide superior robustness with respect to potential sample variations associated with measurements in the process from which the sample is obtained. In particular, each local model can be more parsimonious than a single global model due to the smaller number of sources of signal variations that it must accommodate. This can make the local model more accurate than the global model when applied to spectra obtained within the range of conditions within which the local model is optimized. Furthermore, each local model can be less sensitive than the global model to perturbations caused by sample variations outside those included in the global calibration set. Examples of such a perturbation include, but are not limited to, interferents inadvertently added to the process stream or a temperature excursion outside the anticipated range. As such, the tiered local model classification approach can be more robust with respect to measurements in a process environment and can provide higher accuracy, for instance in an in situ process application.

As is generally known in the art, the goal of PCA is to simplify a data set so that the statistically significant variation within the set is more easily observable. The first PC is calculated to minimize the total difference between itself and all of the spectra in the set. A new data set is formed for which each new “spectrum” is the difference between the original spectrum and the first PC. A second PC is determined from the new data set in the same way, and the process is repeated until the residual data set only contains noise, as determined statistically.

Partial Least Squares (PLS) can provide the link to the value of the characteristic (e.g., concentration) in the data analysis by incorporating standard values into the calculation. Each PC will have a constant (“score”) associated with it that converts magnitude of the PC to a value. In PLS, the first PC and its score can be chosen so that the overall error in concentration prediction is minimized. The residual spectral set is accompanied by a residual concentration vector, and both are used in the calculation of the second PC (with subsequent iterations, as required). The final overall model is the set of PCs and the associated scores. Application of the model to predict the concentration of a new spectrum is relatively straightforward. The first PC is fit to the data set with a single multiplicative factor, the second PC is fit to the residual, and so forth for all PCs. Each magnitude is multiplied by the appropriate score and the total is summed to provide the final concentration prediction.

Ideally, the number of PCs in any one analysis step will be the same as the number of physical factors which are causing changes in the data set. Typically, over the entire range of relevant conditions in a process, the number of factors is large. For instance, examination of an entire actinide calibration data set (e.g., in determination of Pu concentration in a processing line) has indicated that a single universal model would require 9 or more PCs. However, models with a large number of PCs are less desirable as the models will be more susceptible to prediction errors when new spectra are analyzed. The disclosed methods, which can provide predictions that are more robust, provide a route to divide a calibration set into regions and subregions that individually have fewer sources of variation.

A further benefit of the disclosed tiered classification methods is that a much wider range of analyte concentrations can be accurately measured than would be the case with a global single model analysis. This can be accomplished in one embodiment by a systematic evaluation of recorded spectra to detect and subsequently exclude those regions where higher analyte concentrations may lead to reduced measurement accuracy.

The disclosed methods can be utilized in any number of analyte detection protocols for which data such as available in the absorbance spectra illustrates a multivariate nature. One particular example of an application for which the multivariate analysis approach is in actinide measurement including, without limitation, measurement of plutonium, uranium, neptunium, americium, etc. For instance, the analysis technique can be particularly beneficial in conjunction with a Pu purification process as discussed throughout this application. It should be understood, however, that while much of the following discussion is directed to a plutonium measurement application, the disclosed methods are in no way limited to this particular embodiment, or to actinide or solution measurement in general. The disclosed tiered analysis approach is generalizable to any other systems in which the analysis of chemically complicated systems can be aided by rational division of the overall range of sample conditions into simpler subregions. For instance, the tiered analysis approach is applicable to samples in any phase including gases, liquids, solids, plasmas or combinations of phases.

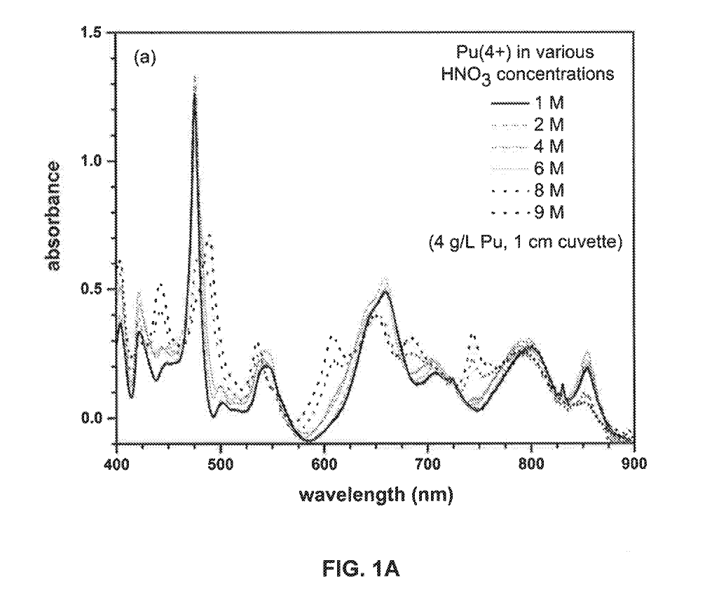

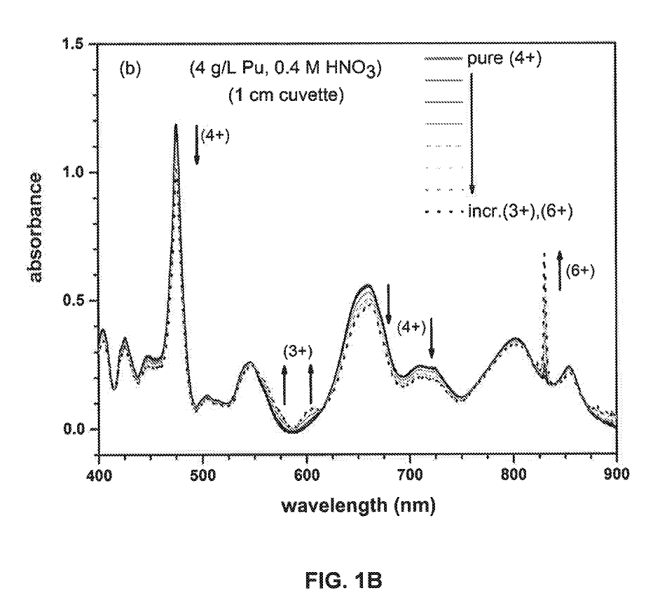

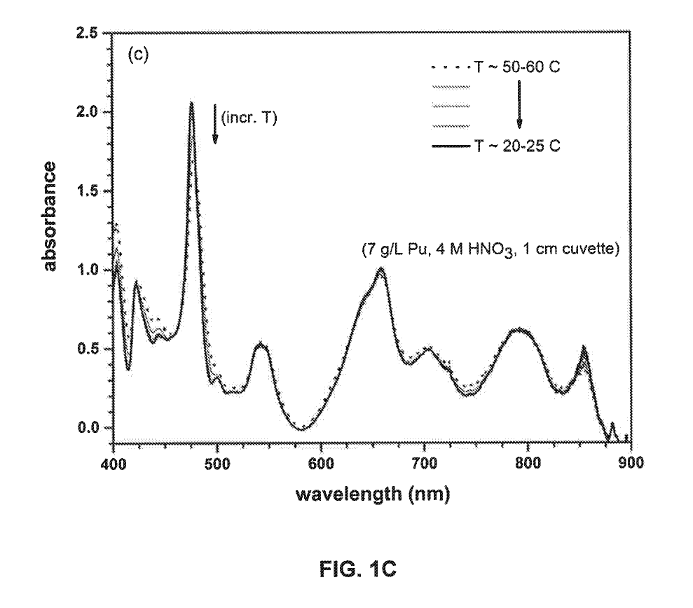

In purifying Pu, on-line, real time measurements of Pu concentration can be used to redirect column eluent to a collection tank and to meet nuclear criticality control levels. To do these tasks, the multivariate model can provide Pu concentration over several orders of magnitude and also should handle significant causes of spectral variability. As shown in FIG. 1A-FIG. 1C, the absorbance spectrum of a Pu solution can significantly vary depending upon the solution acidity (FIG. 1A), the relative concentrations of different Pu oxidation states (FIG. 1B), and the solution temperature (FIG. 1C). The disclosed methods can be particularly useful in such embodiments and can automatically determine accurate concentration levels under such variable conditions without extractive sampling from solutions.

In a PCA, a model can include one principal component (PC) for each non-random source of spectral variation. However, at any specific set of conditions, not all of the sources may be relevant. In such a case, a single global model with all of the PCs appropriate for a full calibration range can effectively overfit a spectrum compared to a model derived from a more limited range of conditions. It is known that overfilling increases the variance of prediction results compared to a more parsimonious model when signal perturbations are introduced. As such, disclosed methods can categorize a broad global spectrum into smaller classes so as to reduce the number of covariant signals and, depending upon the particular spectral classification, provide a local model better suited for increased detection accuracy. Spectral classification can identify specific regions within the total range of process conditions where the solution chemistry is simplified.

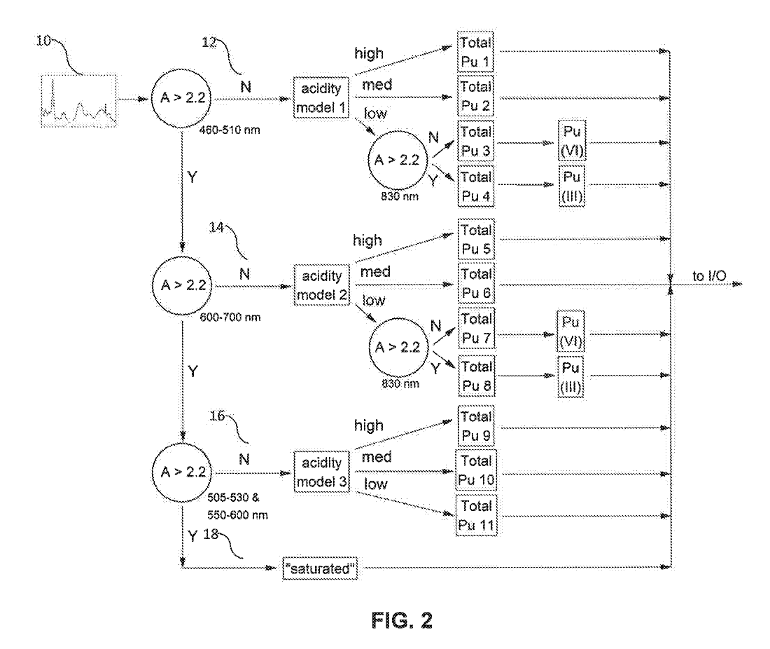

FIG. 2 presents a modeling flowsheet that typifies one embodiment of the disclosed methods. As shown, the modeling flowsheet is designed to categorize an absorption spectrum 10 into a plurality of classes and subclasses within the total range of variable conditions of interest (e.g., concentration, acidity, disproportionation) in which a sample solution may fall. Within each class and subclass, the prediction model can be simpler and more accurate than could be obtained if a single model was used to cover the entire range of conditions,

The flowsheet of FIG. 2 starts at the upper left and generally proceeds across and down. In this particular embodiment, a general absorbance check is performed first to separate the initial spectrum 10 into four different classifications 12, 14, 16, 18, defining the spectrum as “low” 12, “medium” 14, or “high” 16 absorbance, or “saturated” 18. For the saturated class, no further analysis need be done, the total analyte concentration is set equal to the defined maximum value, and a flag can be activated to indicate saturation. Otherwise, additional models appropriate for each absorbance classification can be applied. In this particular embodiment, the second model is directed to the acidity of the sample. If the acidity is determined to be “low” in either the low or medium absorbance classes, then a further absorbance model for that subclass is applied so as to further categorize that subclass according to the secondary sub-classification of analyte oxidation state. This particular scheme of FIG. 2 thus leads to a total of four absorbance classes, nine acidity subclasses, and eleven total analyte models, based on the general and Pu(VI) absorbance and acidity classifications, and two models specific to Pu oxidation state.

The initial classification step in the embodiment of FIG. 2 directed to Pu detection is maximum absorption. The rich set of absorption peaks present for the various Pu oxidation states (FIG. 1A) suggests that even as some peaks exceed the linear range of response of instrumentation, accurate predictions can be made with other peaks. Such as initial wavelength selection can lead to simpler models by avoiding extra PCs required to reproduce nonlinear effects and can expand the dynamic range of a method by allowing measurements at higher concentrations. For example, the range of total plutonium concentration ([Puₜₒₜₐₗ]) that can be measured can be greatly increased (by roughly a factor of 3) by classifying the spectra based on the absorbance at several key wavelengths. As concentration increases, the absorbance at those wavelengths will eventually exceed the threshold for spectrometer accuracy. These wavelengths can be systematically excluded from the data analysis when this occurs by the initial categorization into classes.

The absorbance classification can be applied first to assure that all considered absorbances are in a linear range. Implementing the absorbance criteria for a system can be straightforward. For the particular Pu embodiment of FIG. 2, no more than 25% disproportionation is expected to occur and the majority oxidation state will be Pu⁴⁺, with the upper limit to solution absorbance in this embodiment being 2.2 (though, of course, the threshold can vary depending upon instrumentation, application, etc.). For the example of Pu, the wavelength range that is most likely to have an absorbance above that value for all acidities and is the first to exceed a linearity threshold is between 460-510 nm, which corresponds to the dominant peak in the Pu nitrate spectrum. Thus, upon the first determination, if the absorbance in this wavelength range is below the threshold (e.g., less than 2.2), the solution is categorized into the low absorbance classification 12. If the absorbance is greater than 2.2, then the 600-700 nm region is next evaluated, corresponding to the next largest Pu⁴⁺ peak. A solution not already classified into the low absorbance class 12 that exhibits an absorbance of less than 2.2 in this wavelength range is placed into the medium absorbance classification 14. For solution having a spectrum that has not been categorized into the first or second classes, a third wavelength range (505-530 and 550-600 nm) can be evaluated, with a solution having a spectrum that exhibits an absorbance of less than 2.2 in this range being placed into the high absorbance class 16. Solutions having spectra with excessive absorbance in this region are classified as saturated 18. For this last case, the total analyte concentration is indicated to be greater than the maximum value of the instrument and remaining classifications are undetermined.

The accuracy of prediction from spectra in the high absorbance class 16 can be worse than for the other classes but can still be accurate enough depending on the process monitoring need. As the absorbances in these initial classifications can always scale in the same way, absorbance classification can be logically straightforward, and the maximum absorbance can be simply read off and the spectrum assigned accordingly.

Assuming that the spectrum of the solution is not saturated (i.e., regions 12, 14, and 16), a second step can be carried out that can divide each of the classifications 12, 14, 16, into primary sub-classifications. The second tier of a classification method can be based upon a characteristic of the sample that can be measured at the time of categorization in some embodiments and that can be used to simplify the final prediction measurement model. In the illustrated embodiment, the second categorization step is based upon sample acidity. Acidity is a useful basis in this embodiment because most of the PCs identified in a global Pu model will be associated with nitrate concentration, either directly in the formation of specific nitrato species or indirectly as being a necessary condition for disproportionation. Of course, the second categorization step is not necessarily based upon acidity, and a different sample characteristic may alternatively be utilized, generally depending upon the analyte being detected and the detection conditions.

Each classification can utilize a model designed specifically for that class in order to carry out the second tier division into sub-classes. For instance each absorbance class 12, 14, 16 can utilize a model for acidity that has been designed for that class and that can use acceptable wavelengths to estimate the nitric acid concentration in that class with results divided into three sub-classifications including high (6-9 M), medium (2-6 M) and low (0.3-2M).

The models for each classification can rely on known spectral changes due to the variation of the characteristic of interest and can be provided as input to an analysis process. For instance, acidity within a region can be accurately determined from the spectra, so no additional data (or instruments) are required. This is not a requirement of an analysis however, and in other embodiments, data can be obtained or determined by use of additional or different sources. For instance, in one embodiment, a model can categorize a class or subclass based upon temperature as discussed further herein, and the data utilized in carrying out the model can be obtained from a thermocouple within the process.

In the illustrated embodiment, prediction of the nitric acid content in a classification model can be based on the shapes of the absorption peaks. As shown in FIG. 1A, the most prominent shape change is for the Pu⁴⁺ absorbance peak near 475-490 nm. At lower acidities, the peak is sharp and is centered at 475 nm. As acidity increases, Pu⁴⁺ species with increasing nitrate anions become prevalent. The observed peak becomes the sum of the peaks of the individual Pu nitrate species, with the effect of shifting the center to longer wavelengths and making the peak broader. The equilibrium between the Pu nitrate species, and thus the absorbance spectrum, is independent of total Pu concentration for these process conditions. Since the overall magnitude of the spectrum is dependent on Pu concentration, normalizing the spectra to unit area allows the effects of shape change to be isolated from the effects of Pu concentration.

The transition between Pu⁴⁺ nitrate species with increasing acidity is gradual, with multiple species present at any acidity. The spectral data do not have an intrinsic structure to support statistical classification. Direct determination of the acidity by PLS can provide more flexibility, where the classification criteria can be defined based on the understanding of Pu nitrate chemistry. For example, the higher (certainly 4- and 6-, and possibly 2-) nitrato species do not form below 2M. The establishment of a “low acid” sub-classification below that value can reduce the number of PCs in any model by 2-3. Furthermore, as all disproportionation occurs below 2 M, that criterion removes at least 1 PC from all sub-classifications representing higher acidity. Another categorization border can be established at 6 M, based on the knowledge that the bare and the mononitrate Pu species will not form above that value. Thus, a 3 PC reduction can be expected from a high acid sub-classification defined as about 6M or greater. Even the medium acid subregion between about 2 M and about 6 M can have a 2 PC reduction, as the un-nitrated Pu species is not expected.

Whatever basis is used for dividing each class into multiple subclasses, the basis can be such that within each subclass there will be fewer analyte species than in the entire class. Each of the sub-classifications (e.g., acidity subclasses) can also be chosen so that any further characteristics for categorization (e.g., disproportionation) only occurs in a small number (e.g., one) sub-classification of one or more of the classifications.

Further categorization beyond the first two tiers can be carried out depending upon the particular analyte, sample solution, and/or process conditions. For instance, in an acidic plutonium solution having an acid concentration of about 2 M or less (e.g., [HNO₃]<2M, Pu⁴⁺ disproportionates according to the reaction

3 Pu⁴⁺↔2 Pu³⁺+Pu⁶⁺.

This can lead to monitoring complication due to the fact that the absorption spectra of the Pu nitrates depend on the oxidation state of Pu as illustrated in FIG. 1B. For the spectra of FIG. 1B, [Puₜₒₜₐₗ] and [HNO₃] are constant, and disproportionation is increasing with time. The growth of several peaks due to the increased presence of the (III) and (VI) oxidation states is apparent, with a proportional decrease of peaks attributable to the (IV) oxidation state. Further categorization of the affected acidity sub-classifications for analyte by oxidation state can be of great benefit. For instance, the primary absorbance peak for Pu⁶⁺ at 830 nm is very strong, and there are some scenarios in which it exceeds A=2.2 even if the main Pu⁴⁺ peak does not. Recognition of this situation, if it exists, preserves the accuracy of the determination of total Pu concentration.

The distribution of Pu between the III, IV, and VI oxidation states can be of interest because in most Pu processing applications, separation is specific to the (IV) state. Conversion to the (III) and (VI) states leads to unwanted diversion of Pu to waste streams, and in extreme conditions can lead to issues with criticality safety. Moreover, as is clear by the disproportionation reaction above, tracking the oxidation state distribution can be accomplished by determination of either the III or VI state individually, since they will always appear in a 2:1 state under the conditions of the subregion (acidity less than about 2 M).

Another factor which influences absorption measurements is solution temperature, which is expected to vary between 20-45° C. As shown in FIG. 1C, for some spectrum peaks (475 nm), temperature increases mimic the effect of increasing nitric acid concentration. However, other peaks (650 nm) do not shift similarly, and thus temperature must be considered as a separate influence on the absorption spectrum.

As mentioned previously, categorizations can be based upon additional or different characteristics than those discussed above. For instance, in one embodiment, a spectrum can be classified based upon variation of the process temperature in those embodiments in which temperature variation can lead to spectral changes that are not redundant with those previously utilized.

For example, in the particular case of a Pu analysis, there is a significant decrease of the Pu(IV) 470 nm peak with increasing temperature, but bands at 540, 650, and 800 nm do not change. Temperature variation can influence the spectra through a modification of the equilibrium between plutonium and nitrate (from nitric acid), and it can also influence the spectrum by changing the way the plutonium nitrate complex is surrounded by water molecules in the solution. These two interactions can affect the spectra in different ways, such that a change in temperature does not simply mirror a change in nitric acid content. A decision to further classify based upon a characteristic such as temperature can be application specific. For instance, if analysis is to be carried out over a wide range of temperatures, or if the effects of temperature changes are quite strong, it may be beneficial to incorporate temperature into a classification scheme and make appropriately localized prediction models.

Another basis for classification can include the presence of one or more interferents. For example, interferents such as transition metals can be co-present with Pu and nitric acid in particular segments of a purification process. The presence of interferents can influence a spectrum by causing a large background signal that does not appear equally at all wavelengths. This apparent “color” can challenge the desired absorbance checks and lead to error. According to one method, a method that includes an interferent detection classification step can include analysis of the spectra directly for the interferents, adoption of a specific absorbance classification scheme based on those results, and application of a local prediction model based on the presence (or absence) of the interferents along with the Pu prediction and any other prediction models (e.g., acid prediction).

A classification tier can be based upon the presence of complexants (in addition to or instead of nitrates in a nitric acid solution as discussed previously) are not restricted to nitrate from nitric acid. For instance, oxalate is a common complexant in actinide processing. Organophosphate ligands that can be used in uranium processing are also known and can be used through analysis of spectral characteristics.

Other characteristics that can affect spectral data and be utilized in an analysis process can include the presence of other analytes of interest. For example, an actinide processing stream can include additional actinides (e.g. Pu and U), and the absorbance spectra of the analytes can interfere with one another. Through utilization of the disclosed methods, the concentration or presence of multiple species can be determined through application of different models for each, for instance, depending on an estimate of their relative concentrations. For example, a less precise determination can be a basis to select a particular model for a second, more accurate determination.

Upon carrying out a final categorization (e.g., secondary sub-classification based upon oxidation state of the analyte), a particular PC measurement model for each final sub-classification can be utilized to provide a more accurate determination of analyte concentration in the sub-classification.

In addition to concentration values, all prediction models for total and individual species can generate an evaluation of the spectral fit residuals. This value can be utilized for instance as a measure of how closely the input spectrum resembles the spectra used to make the various prediction models (e.g., spectra generated in the laboratory) used during the analysis. An observation of large residuals can indicate the presence of interfering species in the sample solution, although high solution turbulence can also be a cause.

Among other benefits, disclosed methods can provide an approach to further characterize the sample with additional prediction models, selectively applying those models only in cases where they would be relevant. For example, the concentrations of individual oxidation states of Pu can be determined by use of appropriate PLS models. However, by use of the disclosed methods, these need only be applied for samples that have been classified as low acidity, as it is only under those conditions that the disproportionation reaction is expected to be significant.

The present disclosure may be better understood with reference to the Example set forth below.

### EXAMPLE

The performance of a global PLS model for absorbance spectra of a Pu nitrate system was compared to that of a series of local PLS models that were applied only to a subset of the total range of expected conditions. The appropriate subset was chosen by tiered classifications based on solution absorbance, acidity, and extent of disproportionation. These classifications were determined by separate analyses of the same sample spectra analyzed for the total Pu determination. The models determined total Pu concentration without distinguishing between oxidation states. PLS models were also developed for Pu⁶⁺ and Pu³⁺ that were used to determine the extent of disproportionation by comparing the results of these models to the determined value for total Pu concentration.

### Calibration Standards

Pu⁴⁺ stock solution in nitric acid (HNO₃) was purified and concentrated on an anion exchange column. HNO₃ stock solution was prepared from ACS reagent grade 70% nitric acid. Pu⁴⁺ calibration and validation standards were made gravimetrically from these stock solutions and water. The Pu and nitric acid concentrations in the stock solutions were determined by coulometry and titration, respectively. Pu was confirmed to be present as >99% Pu⁴⁺ in the stock solution by spectrophotometry, specifically by the absence of absorptions near 830 nm that are characteristic of Pu⁶⁺. After mixing, solutions were transferred to 1 cm quartz cuvettes and sealed with polymer-coated screw caps to prevent evaporation. Total 1 a uncertainties of the standards were 0.54% for [Pu] and ˜10% for [NO₃₋]. The uncertainties were based on the combined uncertainties associated with the stock solutions and with those of the balance and densitometer used to track dilutions gravimetrically. A total of 22 calibration solutions were prepared, covering a range of 0-7 g/L Pu and 0.3-9 M HNO₃. Five additional solutions within this range were made up as validation solutions, and the original calibration solutions were remeasured several months after the original calibration solutions to provide additional validation. Seven of these solutions, with acidity ≤2 M, were observed to disproportionate, providing spectra for oxidation state mixtures. Validation solutions for Pu³⁺ and Pu⁶⁺ were made by quantitative reduction or oxidation by addition of ferrous sulfamate and ceric ammonium nitrate, respectively. The completeness of the conversion was confirmed by absorbance spectroscopy.

### Absorption Measurements

Absorption spectra of the sealed cuvettes were obtained using a custom double-beam diode array spectrophotometer. The instrument contained two spectrometers (AvaSpec-ULS3648, Avantes, Broomfield, Colo.), which were configured with a 600 mm⁻¹ grating and 10 μm entrance slit, yielding a spectral resolution of 0.25 nm (based on measurements of lines from a Hg emission lamp) and a pixel resolution of 0.15 nm over 356-916 nm. Optical fibers couple the spectrometers to a cuvette holder inside a radiological glove box. Combined tungsten and Xe arc flashlamp sources provide light over the entire wavelength range of the spectrometer and provide continuous real-time wavelength calibration based on positions of selected Xe emission lines. After wavelength calibration, spectra are interpolated to a common 0.2 nm spacing. The spectrophotometers were corrected for dark current, stray light, and charge readout nonlinearity. Absorbances were calculated from the wavelength- and intensity-corrected raw spectra. Absorbance accuracy was confirmed by measurement of NIST-traceable metal oxide absorbance standards (Firefly Scientific, Brooklyn, N.Y.).

The absorbance response was found to be linear to A>2.2. This value was chosen as an upper threshold for response linearity for the classification models described below. Temperature dependent absorbance spectra were obtained for 6 solutions by heating sealed cuvettes to 60° C. in a heating block, quickly transferring them to the holder, and recording spectra as the cuvettes cooled until the spectra stopped changing. For solutions where there was no disproportionation, the spectra after cooling were identical to those obtained before cooling, indicating that the cuvettes did not leak and that they returned to ambient temperature. The maximum temperature for which spectra were obtained is estimated to be at least 50° C. Spectra for low acidity heated solutions were obtained for an extended period of time, representing different levels of disproportionation, but were not followed to equilibrium due to time constraints.

### Chemometric Analysis

Spectra were analyzed using commercial software (PLS Toolbox, Version 7.5.2, Eigenvector Research, Inc, Wenatchee, Wash.; run within MATLAB, Release R2013a, Mathworks, Natick, Mass.). PLS was used for all quantitation and classification models. Wavelength ranges, noted in the discussions for each model, were based on classification results. All included wavelengths were equally weighted. Except where noted, spectra were transformed to the second derivative using the Savitsky-Golay method. Use of the second derivative addresses potential issues with baseline offsets in the process environment and was consistent with other applications of process absorption spectroscopy. The window sizes used (10-15 nm) retained the character of the distinctively sharp spectral features present at lower acidities. For all models, both the spectra and concentrations were mean centered. Multiple blank spectra of distilled water and nitric acid were included in the Pu calibration sets. They were not included in the acidity models, as is discussed below. Several measures were used to assess the models. The root mean square (RMS) error of cross-validation (RMSECV) was calculated by the average results obtained from random division of the data into 8-10 cross-validation sets and three iterations of the division process. Marginal improvement of the RMSECV with an additional PC was considered indicative of a decreased need for that PC. A more pronounced increase in the ratio of the RMSECV to the RMS error of prediction (RMSEP) for the full calibration set was observed for PCs that were modeling noise instead of signal variation. The signal-to-noise ratio (SNR) for individual PCs was estimated as a feature of the commercial software. Where it is reported, the limit of detection (LOD) was based on three times the standard deviation of results for the blank samples. The models were validated using independent Pu nitrate standards not included in the original model development. These solutions were measured several months after the calibration solutions and on three different spectrophotometers of the same design. All three instruments were calibrated for wavelength and absorbance as described above, and the models were used directly without application of a calibration transfer matrix.

### Global Model for Total Pu

There were seven expected sources of spectral variation for this system, which was expected to lead to seven PCs in a global PLS model. The model obtained for this system met this expectation. The wavelength range of the model was 420-850 nm, covering the significant absorbances for the three Pu oxidation states, with second derivative processing using a 10 nm window and a 3rd-order polynomial. Fit quality parameters are summarized in Table 1 below for models using five to eight PCs, bracketing the expected number. The results provide statistical support for the selection of a 7-PC model, in that the RMSECV, RMSECV/RMSEP ratio (abbreviated CV/P in Table 1), and SNR degrade substantially between the 7th and 8th PCs,

TABLE 1

| | | | | RMSECV | | | LOD | | σ | σᵥₐₗ |
|---|---|---|---|---|---|---|---|---|---|---|
| Model^{(a)} | | | PC^{(b)} | (g/L) | CV/P | SNR^{(c)} | (g/L)^{(d)} | λ (nm) | (%) | (%) |
| Global | | | 5 | 0.17 | 1.072 | 56 | 0.08 | 400-850 | 2.0 | 8.4 |
| | | | 6 | 0.092 | 1.095 | 7 | | | | |
| | | | 7* | 0.083 | 1.085 | 7 | | | | |
| | | | 8 | 0.075 | 1.141 | 3 | | | | |
| Low absorbance | High | 1 | 3 | 0.28 | 1.123 | 145 | 0.05 | 400-850 | 0.8 | 1.1 |
| | acid | | 4* | 0.06 | 1.109 | 13 | | | | |
| | | | 5 | 0.04 | 1.281 | 1.5 | | | | |
| | Med. | 2 | 4 | 0.18 | 1.199 | 22 | 0.04 | 400-850 | 1.3 | 1.0 |
| | acid | | 5* | 0.10 | 1.185 | 7 | | | | |
| | | | 6 | 0.07 | 1.508 | 4 | | | | |
| | (e) | 3 | 3 | 0.24 | 2.021 | 45 | 0.02 | 400-850 | 1.4 | 1.4 |
| | | | 4* | 0.11 | 1.130 | 5 | | | | |
| | | | 5 | 0.09 | 1.315 | 1.9 | | | | |
| | (f) | 4 | 4 | 0.14 | 1.148 | 35 | 0.02 | 400-800 | 2.1 | 3.0 |
| Med. abs. | High | 5 | 4 | 0.11 | 1.404 | 19 | — | 525-850 | 0.9 | 1.8 |
| | acid | | | | | | | | | |
| | Med. | 6 | 5 | 0.12 | 1.377 | 7 | — | | 1.0 | 1.2 |
| | acid | | | | | | | | | |
| | (e) | 7 | 4 | 0.11 | 1.392 | 32 | — | | 1.1 | 3.6 |
| | (f) | 8 | 4 | 0.12 | 1.299 | 63 | — | 525-800 | 1.8 | 2.5 |
| High abs. | High | 9 | 4 | 0.19 | 1.114 | 5 | — | 505-530 + | 0.7 | 1.2 |
| | acid | | | | | | | 550-600 | | |
| | Med. | 10 | 5 | 0.22 | 1.211 | 12 | — | | 1.8 | 1.8 |
| | acid | | | | | | | | | |
| | Low | 11 | 4 | 0.33 | 1.092 | 49 | — | | 1.5 | 2.8 |
| | acid | | | | | | | | | |

| | | | | | | | | |
|---|---|---|---|---|---|---|---|---|
| Pu(VI) | 1 | 0.0004 | 1.02 | 4400 | 0.01 | 800-850 | 0.4 | 3-5 |
| Pu(III) | 3 | 0.02 | 1.07 | 46 | — | 520-650 | 4.3 | |

^{(a)}Labels 1-11 for local Total Pu models correspond to the models indicated in FIG. 2.
^{(b)}For cases where multiple models are compared, the number of PCs chosen is denoted with an asterisk (*).
^{(c)}Estimated signal to noise ratio for the last PC used in the model.
^{(d)}LOD is only shown for low absorbance case models.
^{(e)}Low acidity and low disproportionation (A_{830 nm} ≤2.2).
^{(f)}Low acidity and high disproportionation (A_{830 nm} >2.2).

Within the calibration set, prediction errors were 0.3±1.4% (bias/1σ) for samples ≥6 M, 0.8±2.4% for 2-6 M, and 0.0±2.9% for samples ≤2 M. Larger errors at lower acidity could arise from the comparatively small amount of signal associated with the Pu⁶⁺ peak over the entire wavelength range of the model. The LOD is 0.08 g/L and the estimated upper limit is 7 g/L (these solutions resulted in a peak absorption of 2.2 in the 470-490 nm range), yielding a method dynamic range of 7/0.08=83x. The model did not display a systematic dependence between prediction error and temperature. In contrast, the prediction of heated solution spectra with a 6-PC PLS model based solely on room temperature data yielded errors of (−5)-(−15)% at 50° C.

Prediction errors for the validation set were 8.4%, which is substantially degraded compared to the reproduction of the calibration set. The most significant contributions to the error came from two sources. There was an average variation of 4.0% between the three nominally identical, wavelength- and absorbance-calibrated spectrometers used to measure the validation solution spectra. And, there were large prediction errors ((−10)-(−25)%) associated with the lowest acidity (0.3-0.4 M) solutions. These dependences were consistent with the possibility of local overfitting leading to larger variances under conditions outside, or at the extreme of, those associated with the calibration.

### Acidity Classification Models

Nitrate (equivalently, acidity) models for plutonium nitrate solutions were developed similar to those known in uranyl nitrate system. As the PLS output is proportional to signal magnitude, the spectra must be normalized to Pu concentration in order to isolate the changes due to acidity. However, since the Pu concentration will not be determined until after this classification step, a suitable proxy had to be found. As such, integrated signal was used as a normalization factor. Nitrate was in great excess (e.g. 2 M versus<30 mM Pu) and the distribution of the nitrato complexes (and thus the spectral shape) was effectively independent of the actinide concentration for a given acidity. The integration took place after derivatization (described below) and before mean centering. Because the Pu nitrate absorbance was used to infer nitrate content, it was not possible to use blank solutions (either water or nitric acid) in the calibration set.

On the other hand, since nitrate will always be present with Pu, there was no need to measure blank spectra to determine a LOD for nitrate.

The fitting parameters and prediction results for the three acidity models are shown in Table 2, below. Because these fits were performed before absorbance classification around the Pu⁶⁺ peak at 830 nm, that region of the spectrum was excluded from the initial fits. The second derivative (20 nm window, 5th-order polynomial) was used for the low and medium absorbance cases. The first derivative (15 nm window, 5th-order polynomial) was used for the high absorbance case because there were no discrete peaks in that wavelength range and the second derivatives tended to suppress the differences between spectra. For this case, models based on second derivative spectra gave poor results regardless of the processing parameters chosen. All three models were found to work best with 7 PCs, in agreement with expectations.

TABLE 2

| Abs case | PCs | RMSECV (M) | CV/P | SNR | λ (nm) |
|---|---|---|---|---|---|
| Low | 7 | 0.23 | 1.088 | 15 | 460-750 |
| Medium | 7 | 0.18 | 1.201 | 27 | 525-750 |
| High | 7 | 0.52 | 1.174 | 11 | 505-530 + |
| | | | | | 550-600 |

The RMSECV for the low (0.23 M) and medium (0.18 M) absorbance cases were similar, which was consistent with the expectation associated with both having a large amount of spectral data available. The slightly poorer performance of the low absorbance model was believed to reflect an increased sensitivity of the normalization to disproportionation, which affected the strong 470-490 nm Pu⁴⁺ peak the most. The prediction errors of both models were adequate for the purposes of classification. Of particular interest was the classification border at 2 M, where misclassification of a low acidity solution as medium acidity would specify a different pathway in the classification flowsheet (FIG. 2) and preclude applying a Pu total model that would include disproportionation. However, it was observed that the rate of that reaction was much slower above 1 M than below, and it was not likely for the given process conditions that the instrument would encounter a 2 M solution with disproportionation. Therefore, the consequences of misclassification were not high. Misclassification around 6 M was less critical because disproportionation will not occur in either the medium or high acidity classes, and those classes share several nitrato species. As expected, the performance of the high absorbance model (RMSECV=0.52 M) was comparatively poor due to the small amount of spectral information. However, because the analysis scheme did not include disproportionation for these solutions, a higher uncertainty was acceptable,

Within each model, the absolute prediction error was consistent across the acidity range. Variation due to temperature was also suppressed, especially compared to models generated from room temperature data and applied to spectra of heated solutions. Those models tended to give large errors (nearly +3M for the hottest (50° C.) solutions), which could have significant effects for the analysis of low acidity solutions.

### Local Models for Total Pu

A total of 11 local PLS models for total Pu were generated, per the scheme in FIG. 2. The low absorbance cases (high, medium, and low acidity, with low disproportionation in the latter) were processed in the same way as the global PLS model. The remaining models were calculated with similar preprocessing, but over a truncated wavelength range appropriate for the absorbance classification, as noted in Table 1. Standard spectra of the appropriate acidities were used for each model. Specifically, acidity was ≥6 M for the high acidity models, between 2-6 M (inclusive) for the medium acidity models, and ≤2 M for the low acidity models. The spectral sets overlapped at the 2 M and 6 M boundary conditions to limit the effect of misclassification by promoting consistent analysis of “borderline” spectra by local models from adjoining classification regions.

A summary of the fitting results is shown in Table 1. Generally, the number of PCs used for each model matched the expected number of sources of spectral variation for the smaller acidity range. For illustration of the statistical support of the number of PCs chosen, fit parameters are shown for the low absorbance cases for models using one fewer or one greater PC than the selected number. The same patterns for RMSECV, CV/P ratio, and PC signal-to-noise were observed here as were observed for the global total Pu model.

The one case where a different number of PCs might be expected is for low acidity. A PCA analysis has indicated that there are three Pu⁴⁺ nitrate species below 2 M, which would lead to a total of 5 PCs for the model (temperature and disproportionation providing the other 2 PCs). However, in this analysis the third PC is associated with only 0.17% of the spectral variance, suggesting that the third species is only present in trace quantities in this acidity region. In this case, it is reasonable that the small variations associated with the third nitrate species (presumably the dinitrato complex) cannot be extracted from the data.

Table 1 also shows the model performance for validation solutions, which was close to that for calibration self-prediction. There were several contrasts with respect to the global model. The accuracy for all three instruments represented in the validation set was essentially the same. There was also no dependence on solution acidity for any of the local models within the range for which they were designed. Note that the same validation spectra were analyzed with each set of models, so the differences were not due to experimental artifacts. Instead, the local models were parsimonious with respect to the number of signal variances present for a given solution, and thus were more robust than the larger global model when analyzing spectra outside the calibration set.

The consequence of misclassification near the classification boundaries can be determined by observing prediction errors associated with the total Pu models that would be invoked in such a situation. Table 3, below, compares the performance of adjacent local models for standards in the calibration set with acidities that were nearest the borders. For the 2 and 6 M standards which were used in the models for adjacent regions, the competing models were indistinguishable. With respect to acidity misclassification, some errors became apparent when applying a model to a solution outside its calibration range. These results are shown in brackets in the table. The 2σ uncertainty of the acidity predictions indicates that misclassification is possible within ˜0.5 M of the acidity classification boundary. Therefore, the errors shown in the brackets of Table 3 represent an upper bound to the possible errors that might be observed.

TABLE 3

| Acidity (M) | N^{(a)} | “High”^{(b)} | “Medium” | “Low” |
|---|---|---|---|---|
| 7 | 14 | 1.0 ± 0.7% | [−7.6 ± 0.8%] | — |
| 6 | 14 | 0.0 ± 2.8% | 0.6 ± 2.4% | — |
| 5 | 14 | [−3.8 ± 2.1%] | 0.1 ± 1.1% | — |
| 3 | 4 | — | 1.0 ± 3.3% | [5.0 ± 3.7%] |
| 2 | 12 | — | −0.5 ± 1.9% | −0.2 ± 2.0% |
| 1 | 7 | — | [−0.2 ± 3.1%] | −0.6 ±3.0% |

^{(a)}N = number of samples.
^{(b)}Values in brackets represent the results of applying a model outside the intended acidity range. For example, a “high” acidity model would ideally not be applied to a solution with an acidity of 5M.

The total dynamic range of the method was determined by comparing the LODs for the low absorbance models and the maximum concentration measurable with the high absorbance models. For example, for 6 M solutions, these quantities were 0.05 and 32 g/L, respectively. This yielded a dynamic range of ˜640×(2.8 orders of magnitude). The dynamic range was acid dependent, due to changes in both the LOD and the upper limit. The range was smallest for 6 M solution, and was largest for 1-2 M solutions (33 g/L/0.02 g/L=1650×).

### Comparison of the Tiered and Global PLS Analyses

When considered in their ability to reproduce the concentrations of the calibration sets, the tiered, local PLS model approach and the global PLS model performed equivalently. This was expected, since the spectra were of high quality (low noise, linear response) and the system was well-behaved chemically (absorbances were linear with [Pu]). One clear advantage of the tiered local model approach for the calibration set was the expansion of the dynamic range by about one order of magnitude through the systematic exclusion of wavelengths with excessive absorbance. A justification for using the tiered approach comes from the results obtained when analyzing spectra outside the calibration set.

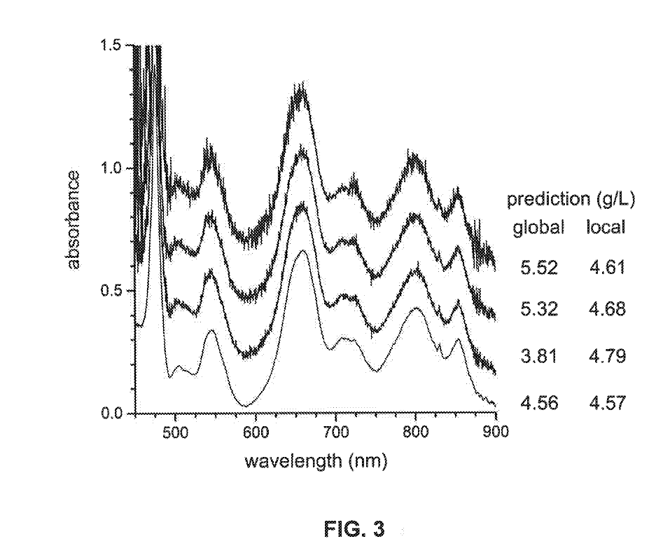

The effects of instrument noise were explored with the analyses of spectra of the same solutions with different spectrometers. A second analysis more pertinent to field operation gives similar results. FIG. 3 shows a simulated data set in which successively larger amounts of white noise were added to a spectrum obtained during column elution (spectra are offset for clarity). These spectra were analyzed with both the global Pu PLS model and the appropriate (low absorbance, low acidity) local Pu model within the tiered scheme. For the spectrum with no noise added, the two models gave the same result. However, the local model was much more tolerant of the added noise than the global model. The noise was representative of the effects of electrical line noise, lamp instability, and other conditions typical of the process environment. The improved robustness of the local models in the tiered scheme was apparent.

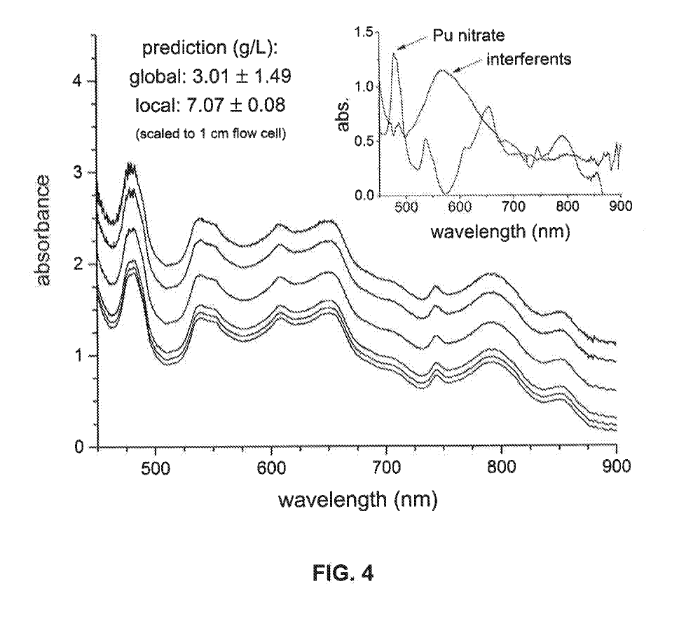

Another situation common in process measurements is the presence of unanticipated interferents. FIG. 4 shows several spectra in which the Pu nitrate solution also contained unknown amounts of Fe, Cr, and Ni. These spectra were observed during a stage of the process that was outside the scope of the intended use of the process monitor. Thus, the transition metals were not included in the calibration. These spectra are not offset for clarity. Baseline variation was due to different amounts of solution turbulence from entrained air. The robustness of the local model approach is seen in the relative consistency of the output. The comparison of the different measurement results was facilitated by analysis of the spectra shown in the insert. Here, one of the process spectra was deconstructed to the spectrum of a solution from the calibration set (Pu at 8 M HNO₃) and an “interferent” spectrum that is the difference of the process and calibration solutions. The global and local Pu models gave similar results (within 0.6%) for the calibration solution. However, the global model gives a large negative result (−4 g/L) for the interferent spectrum, while the local model response was approximately zero and the result for the original solution measurement was close to the expected value.

Models for Pu⁶⁺ and Pu³⁺

Because the disproportionating solutions were not at equilibrium, any aliquots removed from the solution would continue to change before a confirmatory independent analysis could be performed. Thus, the concentrations of the Pu³⁺ and Pu⁶⁺ were estimated from the absorbance spectra using a constrained PCA method. Each solution generated a series of spectra that were linear combinations of a Pu⁴⁺ component and “2 Pu³⁺+Pu⁶⁺” component, the latter itself being the combination of spectra of those two oxidation states. The analysis was performed individually for each of 7 standards covering 0.3-0.7 M and 4-7 g/L Pu. For each standard tested, only 2 PCs were indicated.

The two PCs obtained were linear combinations of the Pu⁴⁺ and Pu³⁺/Pu⁶⁺ components. Because they are orthogonal, the original PCs represent the basis for a two-dimensional space. The Pu⁴⁺ and Pu³⁺/Pu⁶⁺ vectors, being orthogonal to each other, also form a basis set for this space. The relationship between the two sets is defined as an angle of rotation, θ, which can be determined with an additional constraint.

The Pu⁶⁺ second derivative spectrum near 830 nm is a strong signal, while the Pu⁴⁺ spectrum is nearly flat. Thus, rotating the PCs to minimize the signal in the 800-850 nm region for one of the PCs will approximate the Pu⁴⁺ spectrum. Simultaneously, the second PC will approximate the combined Pu³⁺/Pu⁶⁺ spectrum. Mathematically, this is accomplished by minimizing the magnitude of the rotated sub-vector,

[symbol]

[(PC) [symbol] _{(1,rot)}]²=cos² θ· [symbol] [PC [symbol] ₁]²+sin² θ· [symbol] [PC [symbol] ₂]²+2 cos θ sin θ [symbol] [PC [symbol] ₁] [symbol] [PC [symbol] ₂]

where the vector computations are carried out only over the range 800-850 nm.

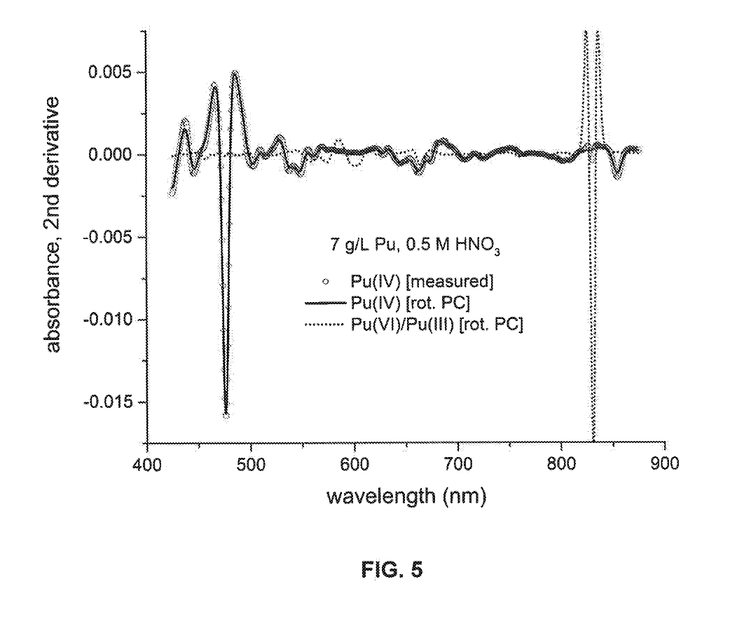

Typical results are shown in FIG. 5. A very close overlap was seen between the Pu⁴⁺ second derivative spectrum for a standard solution at 0.5 M HNO₃ (circles) and the deduced spectrum obtained by rotation (solid line). The agreement, which was obtained without fitting to the standard spectrum, supported the validity of this approach. Further confirmation was obtained when the above calculation was performed by using the criterion of minimizing [PC_{2,rot}]² in the region of the Pu⁴⁺ peak at 475 nm. When calculated this way, the same rotation angle and rotated PCs were obtained.

The approach is a self-consistent method because the calculations do not include a fit to the pure spectrum of either components or require a priori knowledge of the component concentrations at any time. As the cuvettes containing these solutions were sealed, the total Pu concentration remained constant, allowing all of the spectra in a set to be compared without normalization. This approach is especially useful for analysis of those lowest-acidity solutions where disproportionation starts immediately upon mixing and it is not possible to measure a “time=0” spectrum of pure Pu⁴⁺.

Once the scores for the rotated PCs were obtained, the concentrations of the disproportionation products were calculated for each spectrum. Of the Pu that was not in the IV oxidation state, ⅔rd is Pu³⁺ and ⅓rd is Pu⁶⁺. With these concentrations, PLS models for Pu⁶⁺ and Pu³⁺ were generated in the same manner as for the total Pu models. Fit parameters and quality metrics are shown in Table 1. The Pu⁶⁺ model required only 1 PC and was very accurate. However, as the Pu⁶⁺ used in this model concentrations was also obtained by PCA with a single PC, this accuracy primarily reflected the ability of the model to reproduce itself (the errors in the approach are all in the concentration estimates). The validation results, presented below, are more representative of the accuracy of the model. The LOD for this model was 0.01 g/L (1 cm path length); the lower number compared to Pu⁴⁺ reflected the larger absorptivity for Pu⁶⁺. The Pu³⁺ model required 3 PCs, consistent with a greater influence of both acidity and temperature. The higher self-prediction errors observed were probably more representative of the true predictive capability. Neither the Pu⁶⁺ nor the Pu³⁺ model used the 460-510 nm region of the spectrum. Therefore, these models were applied to spectra that had passed either the first (460-510 nm) or the second (600-700 nm) global absorbance checks.

One independent measure of the true accuracy of the Pu⁶⁺ PLS model was obtained from the analysis of spectra of solutions where Pu was quantitatively oxidized to the VI state using ceric ammonium nitrate. A total of 11 solutions were thus prepared with acidities between 1-2 M (one solution had an acidity of 0.3 M) and Pu concentrations of 0.02-0.8 g/L. The ceric ammonium nitrate was not expected to cause any chemical or spectral interferences with the Pu⁴⁺ peak, and therefore the PLS model would be expected to be relevant to these solutions. Replicate spectra were obtained of each solution on separate days. Application of the PLS model resulted in a prediction error of 5.3%, with a bias of −1.9%. Fit errors for spectra of the same solution acquired on different days were the same within ˜1%.

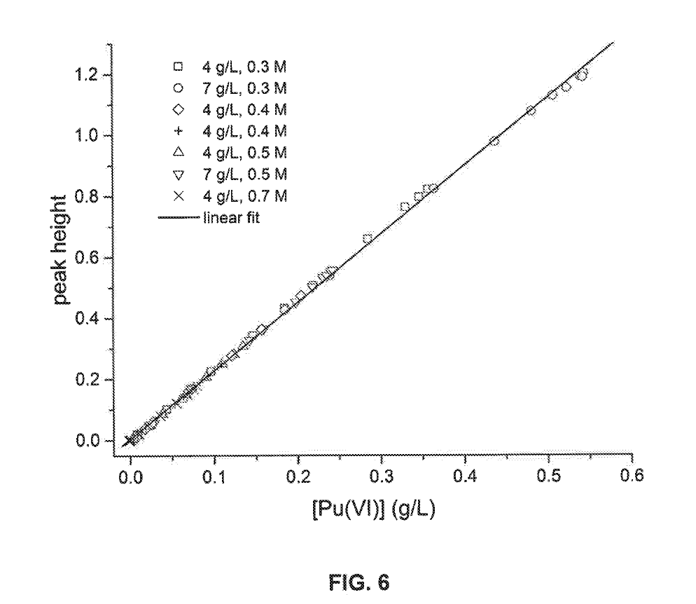

A second measure of the accuracy was made by comparing the deduced molar absorptivity of Pu⁶⁺ to the literature value ε˜550 M⁻¹ cm⁻¹, obtained for a spectral resolution of 0.2 nm (versus 0.25 nm in this study). The height of the Pu⁶⁺ peak in each spectrum was determined by subtracting a baseline value (average of ˜825 and 835 nm) from the peak maximum. As shown in FIG. 6, peak heights were plotted against the derived Pu⁶⁺ concentration from the PCA analysis; the slope of the linear fit of these points is equivalent to the absorptivity. There was no apparent dependence of the slope on [Pu] or acidity, and thus all standards were pooled for a single fit. Assuming an atomic mass of 239.1 g/mole, the slope yielded a molar absorptivity of 542(14) M⁻¹ cm⁻¹, which is consistent with the literature value. The relative uncertainty of 2.6% was similar to the value obtained from the quantitative oxidation. These errors were also consistent with those observed for the Pu³⁺ model. Considering that the original concentration estimates for the (III) and (VI) species were obtained without any information about the solutions apart from the total Pu concentration, the errors observed for these validation studies confirmed that the self-consistent PCA analysis is a suitable method for developing PLS models for Pu disproportionation measurements.

While certain embodiments of the disclosed subject matter have been described using specific terms, such description is for illustrative purposes only, and it is to be understood that changes and variations may be made without departing from the spirit or scope of the subject matter.

What is claimed is:

1. A method for analyzing a sample comprising:
assigning a sample into one of multiple classifications based upon a value of a first characteristic of the sample, the value of the first characteristic being determined through the interpretation of multivariate data, wherein values of the multivariate data are sensitive to a concentration of an analyte contained in the sample and/or are sensitive to a concentration of one or more additional components of the sample and/or are sensitive to a property of the sample;
further assigning the sample into one of multiple primary sub-classifications based upon a value of a second characteristic of the sample, the value of the second characteristic being determined through further interpretation of the multivariate data;
optionally, further categorizing the sample into one of multiple secondary sub-classifications based upon a value of a third characteristic of the sample; and
predicting the concentration of the analyte in the sample through application of one or more measurement models specific to the categorizations of classification, primary sub-classification and optional secondary sub-classification of the sample.

2. The method of claim 1, wherein the multivariate data is generated by use of multiple instruments.

3. The method of claim 1, wherein the multivariate data is generated by use of a single instrument.

4. The method of claim 1, wherein the sample is in the form of a solution.

5. The method of claim 1, wherein at least a portion of the multivariate data is generated by spectral analysis.

6. The method of claim 1, wherein at least one of the first, second, and third characteristics is absorbance of the solution at one or more pre-determined wavelength ranges.

7. The method of claim 1, wherein at least one of the first, second, and third characteristics is solution acidity or oxidation state of the analyte.

8. The method of claim 1, wherein the sample is further categorized into secondary sub-classifications for only a portion of the primary sub-classifications.

9. The method of claim 1, wherein one or more of the first, second, and third characteristics comprises temperature, the presence of an interferent, complexation, the presence of a second analyte, or the presence of a complexant.

10. The method of claim 1, wherein the analyte comprises an actinide.

11. The method of claim 1, wherein the method is carried out in-line in a processing stream.

12. The method of claim 1, wherein the method comprises a principal component type analysis.

13. The method of claim 12, wherein the method comprises a partial least squares analysis.

14. A method for determining the presence or concentration of an actinide in a solution, the method comprising:
assigning the solution into one of multiple classifications based upon the absorbance of the solution at one or more wavelength ranges;
further assigning the solution into one of multiple primary sub-classifications based upon a value of a first characteristic of the solution, the value of the first characteristic being determined through the interpretation of an absorbance spectrum of the solution, wherein values of the absorbance spectrum are sensitive to the concentration of the actinide in the solution and/or are sensitive to a concentration of one or more additional components of the solution and/or are sensitive to a property of the solution;
further assigning the solution into one of multiple secondary sub-classifications based upon a value of a second characteristic of the solution, the value of the second characteristic being determined through further interpretation of the absorbance spectrum; and
predicting the concentration of the actinide in the solution through application of one or more measurement models specific to the categorizations of classification, primary sub-classification and optional secondary sub-classification of the solution.

15. The method of claim 14, wherein the first characteristic comprises acidity of the solution.

16. The method of claim 14, wherein the second characteristic comprises oxidation state of the actinide.

17. The method of claim 14, wherein the actinide comprises plutonium.

18. The method of claim 14, wherein the method is carried out in-line in an actinide processing stream.

19. The method of claim 14, wherein the method comprises a principal component type analysis.

20. The method of claim 19, wherein the method comprises a partial least squares analysis.
